(19) United States
(12) Plant Patent Application Publication
(10) Pub. No.: US 2015/0067934 P1
(43) Pub. Date: Mar. 5, 2015

(54) Cherry rootstock plant named 'STO1'

(50) ;

(71) Applicant: Peter Stoppel Obstbau, (US)

(72) Inventor: Peter Stoppel, Kressbronn (DE)

(21) Appl. No.: 13/987,744

(22) Filed: Aug. 27, 2013

(51) Int. Cl. A01H 5/00

(52) U.S. Cl. PLT/183

(57) ABSTRACT

A new and distinct cultivar of Prunus plant named ‘STO1,’ characterized by its upright plant habit and medium strong growth; good adaptation at hot and dry conditions; and as a rootstock the weak vegetative growth imparts uniformity, higher yield and better fertility to the grafted variety.

### LATIN NAME OF THE GENUS AND SPECIES

The Latin name of the genus and species of the plant variety disclosed herein is Prunus×gondouinii (Poit. & Turpin) Rehder (P. avium (L.) L.×P. cerasus L.).

### VARIETY DENOMINATION

The inventive cultivar of Prunus×gondouinii (Poit. & Turpin) Rehder (P. avium (L.) L.×P. cerasus L.) disclosed herein has been given the varietal denomination ‘STO1.’

### BACKGROUND OF THE INVENTION

The present invention relates to a new and distinct cultivar of Prunus plant, typically used as a cherry tree rootstock, botanically known as Prunus×gondouinii (Poit. & Turpin) Rehder (P. avium (L.) L.×P. cerasus L.), and hereinafter referred to by the name ‘STO1.’

The new and distinct cultivar of cherry rootstock of the present invention was bred as a cross of Prunus cerasus with a wild Prunus sp. in Germany in 1989. It has been successfully asexually propagated by tissue culture since the first time in 2002. The purpose of the breeding program was to find better adapted cherry rootstocks. The new Prunus plant is better adapted on heat and aridity than other rootstocks with a weak vegetative growth. The breeder succeeded in creating a new cherry rootstock, which has a higher yield and influences the grafted variety positively in fertility.

### SUMMARY OF THE INVENTION

Plants of the new Prunus have not been observed under all possible environmental conditions. The phenotype may vary slightly with variations in environment such as temperature and light intensity, without any variance in genotype.

The following traits have been repeatedly observed and are determined to be the unique characteristics of ‘STO1.’ These characteristics in combination distinguish ‘STO1,’ as a new and distinct cultivar of Prunus:

1. Upright plant habit;

2. Good adaptation to hot and dry clime conditions;

3. As a rootstock, with weak vegetative growth, it produces a dwarf tree;

4. As a rootstock, imparts uniformity and higher yield to the grafted variety;

and

5. As a rootstock, grafted varieties have a 60% weaker growth than a Prunus avium seedling.

Plants of the new Prunus can be compared to plants of Prunus ‘Weiroot 720’ (U.S. Pat. No. 22,867, issued Jul. 24, 2012). In side-by-side comparisons conducted in Kressbronn, Germany, plants of the new Prunus differed primarily from plants of ‘Weiroot 720’ in the following characteristics:

1. As a rootstock, plants of the new Prunus have a one-third stronger vegetative growth than plants with ‘Weiroot 720’ rootstocks;

2. Plants of the new Prunus develop stipules, whereas ‘Weiroot 720’ hasn't any stipules;

3. Plants of the new Prunus produce larger fruit. Average fruit diameter ‘STO1’:1.9 cm, ‘Weiroot 720’:1.2 cm. Average fruit weight ‘STO1:4 g, ‘Weiroot 720’:1.6 g;

4. Due to its typical/characteristic growth, plants of the new Prunus impart higher yield to the scion plant than plants of ‘Weiroot 720;’ and

5. Fruits from plants of the new Prunus ripen about 10 days earlier than fruits from the ‘Weiroot 720.’

Asexual reproduction of Prunus rootstock plant ‘STO1’ by tissue culture since 2002 has shown that the unique features of this new variety are stable and the plant reproduces true to type in succeeding generations of asexual reproduction.

### BRIEF DESCRIPTION OF THE DRAWINGS

The accompanying photographs illustrate the overall appearance of the new Prunus plant. These photographs show the colors as true as it is reasonably possible to obtain in colored reproductions of this type. Colors in the photographs may differ slightly from the color values cited in the detailed botanical description which accurately describe the colors of the new Prunus plant.

FIG. 1 provides a side perspective view of a typical tree of ‘STO1’ grown in an outdoor orchard.

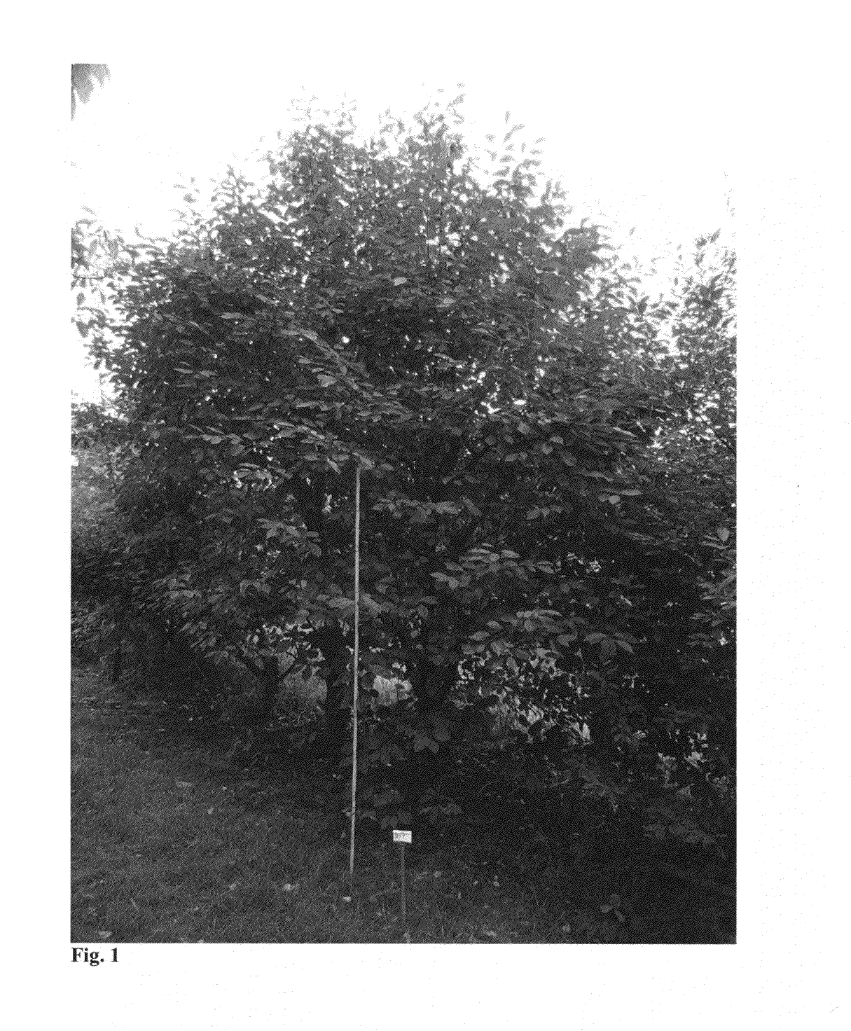

FIG. 2 provides a close-up view of leaves of ‘STO1.’

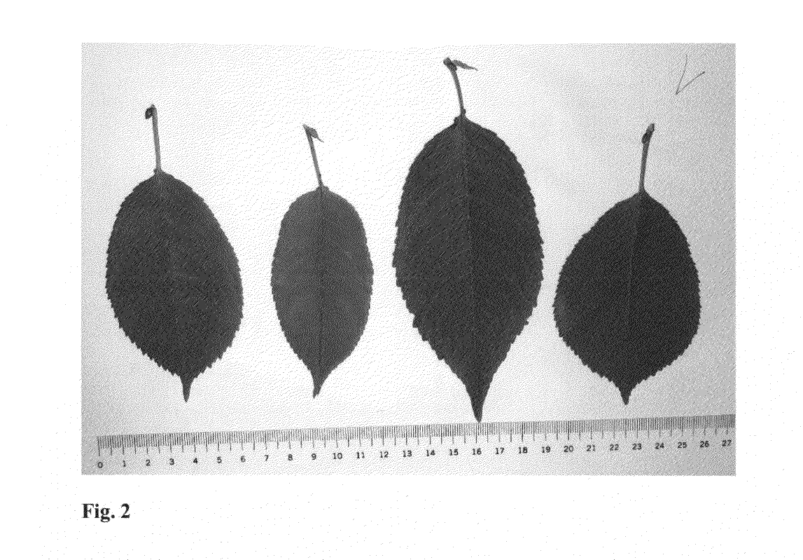

FIG. 3 provides a close-up view of fruits, branches and leaves of ‘STO1’.

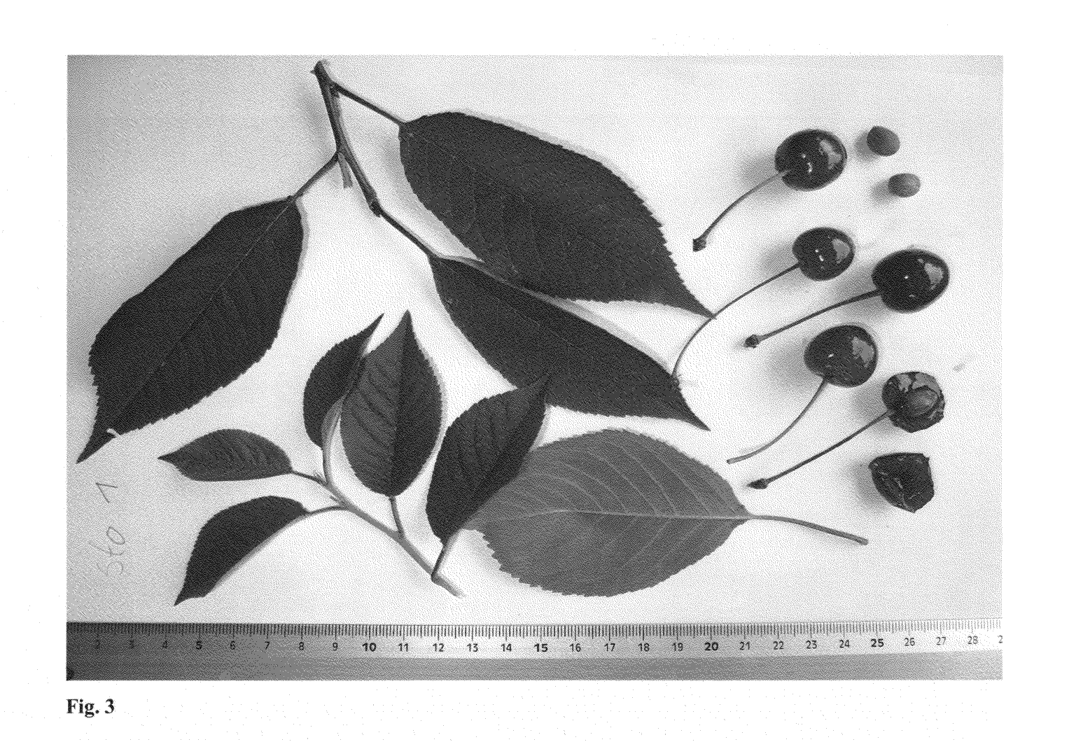

### DETAILED BOTANICAL DESCRIPTION

The aforementioned photographs, following observations and measurements describe plants grown during the summer in Kessbronn, Germany in an outdoor orchard and under conditions and practices generally used in commercial cherry production. Plants were 11 years old when the photographs and descriptions were taken. Measurements and numerical values represent averages for typical plants and plant parts. The actual measurements of any individual plant or plant parts, or any group of plants or plant parts, of the new Prunus plant may vary from the stated average. In the following description, color references are made to The Royal Horticultural Society (R.H.S.) Colour Chart, except where general terms of ordinary dictionary significance are used.

‘STO1’ has a generally erect plant habit and is medium strong in growth. After eleven growing seasons in Kressbronn, Germany, a height of about 4.8 meters, a width of about 3.5 meters and a trunk diameter of about 14 cm were observed for ‘STO1.’ The growth reduction induced by ‘STO1’ allows a highly intensive cherry production with dwarf trees.
Botanical classification: Prunus×gondouinii (Poit. & Turpin) Rehder (P. avium (L.) L.×P. cerasus L.) ‘STO1’
Parentage: Breeding between Prunus cerasus and a Prunus avium species.
Propagation:
   Type.—By asexual reproduction.
   Method.—By tissue culture since 2002 in a controlled environment. The process is similar to the propagation of other cherry rootstocks.
Plant description:
   Plant and growth habit.—Used a rootstock; upright plant habit; plants are typically grown as a single stem; moderate vigor.
   Stem description.—Strength: Strong. Texture: Rough. Color: Grey brownish RHS 199B.
   Leaf description.—Arrangement: Alternate; simple. Length: about 7.3 cm to about 12 cm. Width: about 4 cm to 7 cm. Shape: Ovate, moderately elongated to medium. Apex: Acute. Tip: Acuminate. Base: acute angle, flat curvature. Margin: Biserrate to serrulate. Texture, upper surface: Glabrous. Texture, lower surface: Pilose. Color: Fully developed leaves, upper surface: Close to RHS 137A; venation, close to RHS 141C. Fully developed leaves, lower surface: Close to RHS 137C; venation, close to RSH 161C.
   Petiole description.—Length: about 2 cm to about 3 cm. Diameter: About 0.1 cm to about 0.21 cm. Color, upper surfaces and lower surfaces: Close to RHS 173B.
   Presence of stipules.—Present. Frequency: Not at every bud.
   Stipule description.—Length: About 1.5 cm. Width: About 0.15 cm. Shape: Narrow, very elongated.
Flower description:
   Flower type/habit.—2 to 4 single flowers arranged one compound panicles. Flowers occur in general on one year old branches.
   Fragrance.—Weak, pleasant.
   Natural flowering season.—In April, in south Germany, two days after ‘Gisela 3.’ Three days earlier than ‘Weirood 720.’
   Lastingness of entire bloom period.—About 16 days.
   Lastingness of an individual bloom.—About 10 days.
   Inflorescence height.—About 4.3 cm.
   Inflorescence diameter.—About 4.5 cm.
   Bloom quantity.—Fertile, a large quantity of flowers occur each year.
   Flower diameter.—About 2.8 cm to about 3.1 cm.
   Flower depth.—About 0.6 cm to about 1.5 cm. The pistil is very long, so the stigma overtops the anthers and the petals.
   Petals.—Arrangement: Five petals in a single whorl, intermediate to slightly overlapping. Length: About 1.2 cm Width: About 1.1 cm. Shape: Oblong, slightly elongated. Apex: Rounded, slightly elongated. Base: Truncate. Margin: Entire. Texture, upper and lower surfaces: Smooth, glabrous. Color: When fully opened, upper surface: Close to RHS 155B. When opening and fully opened, lower surface: Close to RHS 155D.
   Sepals.—Arrangement: Five sepals in a single whorl. Length: About 0.5 cm. Width: About 0.28 cm. Shape: Ovate, moderately elongated. Apex: Rounded. Base: Truncate. Margin: Entire. Texture, upper and lower surfaces: Smooth, glabrous. Color, upper surfaces: Close to RHS 142A. Color, lower surfaces: between RHS 134A and RHS 185A.
   Pedicels.—Length: About 2.15 cm. Diameter: About 0.1 cm. Angle: About semi-erect to semi-prostrate from the inflorescence stalk. Texture: Smooth, glabrous. Color: Close to RHS 142A.
   Peduncle.—Length: About 0.2 cm. Diameter: About 0.1 cm Surface: Smooth. Color: Close to RHS 149A.
   Reproductive organs.—Stamens: Quantity per flower: Numerous. Filament length: About 3 mm to about 10 mm. Filament color: Close to RHS 155B. Anther length: About 0.5 mm to about 1.3 mm. Anther color: Close to RHS 6A. Pollen color: Close to RHS 6D. Pistils: Quantity per flower: One. Pistil length: About 9 mm to about 16 mm. Style length: About 8 mm to about 11 mm. Style color: Close to RHS 154B. Stigma shape: Round. Stigma color: Close to RHS 154A.
   Fruit description.—Maturity when described. Date picking: July in south Germany, varies with climatic conditions. About ten days earlier than ‘Weiroot 720’, (U.S. Pat. No. 22,867). Size: small to medium size; about 1.6 cm to about 2.1 cm diameter. Average weight: About 4 g, varies slightly with fertility of the soil, amount of thinning and climatic conditions. Form: Globose, Slightly flat at the apex. Stem cavity: Slightly obcordate to flat. Color: RHS 53A.
   Peduncle description.—Length: about 3.2 cm to about 5.2 cm. Average diameter about 0.09 cm. Color: RHS 179A.
   Stone description.—Average length: About 1 cm. Average width: About 0.8 cm. Average thickness: About 0.65 cm. Form: Nearly ovoid. Base: Flat. Apex: Obtuse. Color: RHS 164B when dry.
   Fruit use.—not be used as a dessert fruit.
Temperature tolerance: Plants of the new Prunus have been observed to tolerate temperatures from about −20° C. to about 40° C.

What is claimed is:

1. A new and distinct cultivar of Prunus×gondouinii (Poit. & Turpin) Rehder (P. avium (L.) L.×P. cerasus L.) named ‘STO1’, substantially as illustrated and described herein.
